(19) United States
(12) Patent Application Publication
(10) Pub. No.: US 2008/0208029 A1
(43) Pub. Date: Aug. 28, 2008

(54) Garment Comprising At Least One Dry Electrode

(75) Inventors: Jeroen Thijs, Aachen (DE); Olaf Such, Aachen (DE); Jens Muehlsteff, Aachen (DE); Ralf Schmidt, Aachen (DE)

Correspondence Address: PHILIPS INTELLECTUAL PROPERTY & STANDARDS, 595 MINER ROAD, CLEVELAND, OH, 44143, US

(73) Assignee: KONINKLIJKE PHILIPS ELECTRONICS N.V., Eindhoven (NL)

(21) Appl. No.: 11/817,925

(22) PCT Filed: Mar. 1, 2006
(86) PCT No.: PCT/IB2006/050631

§ 371 Date: Sep. 6, 2007

(30) Foreign Application Priority Data
Mar. 8, 2005 (EP) 05101782.0

(51) Int. Cl. A61B 5/048

(52) U.S. Cl. 600/388

(57) ABSTRACT

It is an object of the invention to provide a user-friendly monitoring system with improved measurement quality, which is suitable for home use. This object is achieved according to the invention by a garment (1) comprising at least one dry electrode (2, 3, 4) for use with a monitoring system for monitoring physiological parameters of a recipient, the electrode (2, 3, 4) having a working surface which is to be brought into contact with the recipient's skin, characterized in that the working surface is automatically positioned at a point of close skin contact with a natural sweat layer present, if the garment (1) is worn by the recipient. The basic principle underlying the invention is that the position of the electrodes (2, 3, 4) on the body has an impact on the acquired signal quality. Therefore, it is an idea of the invention to arrange dry electrodes in positions of the garment (1) where close body contact is automatically obtained and a natural sweat layer is present, if the garment is worn. Because a natural sweat layer is already present around the armpit, the operability of the electrode (2, 3, 4) is given automatically, if it is located in the armpit area (10).

The present invention relates to a garment comprising at least one dry electrode for use with a non-invasive monitoring system for monitoring physiological parameters of a recipient, the electrode having a working surface which is to be brought into contact with the recipient's skin. Furthermore, the invention relates to a method of monitoring physiological parameters of a recipient, using a garment comprising at least one dry electrode having a working surface which is to be brought into contact with the recipient's skin.

Dry electrodes are directly applied to the recipient's skin without any use of a conductive gel or the like. Because of the easy handling of such electrodes, their user-comfort is clearly enhanced compared to wet electrodes, which are mainly used in clinical applications. However, a disadvantage of dry electrodes is the long warm-up time because of the time necessary for the formation of a sufficient sweat-layer under the electrode. Such a liquid layer is needed to reach an impedance level low enough for a reasonable signal-to-noise level.

US patent application 2004/0138546 A1 suggests to provide a sensor with an additional moisture layer for retaining moisture. Such a moisture layer retains secretory products from the skin. However, employing such a moisture layer leads to a more complicated type of construction and to an increase in production costs.

It is an object of the invention to provide a user-friendly monitoring system with improved measurement quality, which is suitable for home use.

This object is achieved according to the invention by a garment comprising at least one dry electrode for use with a monitoring system for monitoring physiological parameters of a recipient, the electrode having a working surface which is to be brought into contact with the recipient's skin, characterized in that the working surface is automatically positioned at a point of close skin contact with a natural sweat layer present, if the garment is worn by the recipient.

This object is also achieved according to the invention by a method of monitoring physiological parameters of a recipient by means of a non-invasive monitoring system using a garment comprising at least one dry electrode having a working surface which is to be brought into contact with the recipient's skin, characterized in that the working surface is automatically positioned at a point of close skin contact with a natural sweat layer present, if the garment is worn by the recipient.

The basic principle underlying the invention is that the position of the electrodes on the body has an impact on the acquired signal quality. Therefore, it is an idea of the invention to arrange dry electrodes in positions of the garment where close body contact is automatically obtained and a natural sweat layer is present, if the garment is worn. Because of the fact that dry electrodes are used, good wearing comfort is achieved.

The garment can therefore be used for long-term on-body measurements and for home use. With the invention the use of dry electrodes is enhanced for several personal healthcare applications, for example for trend analysis or personal monitoring. A preferred example of an application is a personal monitoring device for long-term ECG (electrocardiographic) monitoring in order to identify possible arrythmias that occur relatively infrequently.

Because the close body contact is achieved immediately after the garment is used and a natural sweat layer is present, the time needed for lowering the impedance by the sweat layer is no longer a limiting factor. With the invention the signal-to-noise ratio can be increased and the warmup time can be decreased significantly. The technique according to the invention is therefore preferred to more traditional dry electrode solutions, such as wearing a belt around the waist or chest.

These and other aspects of the invention will be further elaborated on the basis of the following embodiments, which are defined in the dependent claims.

In a preferred embodiment of the invention, the at least one dry electrode is integrated into the garment. Such an integrated electrode is not only very user-friendly, but also contributes to the comfort of the recipient. Forms of integration are inweaving, sewing, gluing, knitting, embroidering etc.

In another preferred embodiment of the invention, the garment is a shirt or the like, and the at least one dry electrode is located in the armpit area of that shirt, i.e. on the left and/or right side of the upper thorax. Because a natural sweat layer is already present around the armpit, the operability of the electrode is given automatically. The electrode is located at a point on the body where close contact by clothing is normal and accepted. In other words, the use of a dry electrode will not negatively affect the wearing comfort of the shirt, e.g. an underwear shirt, because people are used to wearing clothing that is tight around the arm pits. Such an electrode is simple to use, because it does not need to be wetted separately. The electrode can be attached not only to a shirt or undershirt, but also to various pieces of clothing, such as a brassiere, a top etc.

According to a further embodiment of the invention, the electrodes are ECG electrodes for implementing an ECG measurement. ECG is employed, e.g., for analyzing heart rhythm and heart conditions.

According to still another embodiment of the invention, the electrodes are impedance electrodes for implementing an impedance measurement. Such measurements are employed, e.g., for determining respiration rate and intensity.

These and other aspects of the invention will be described in detail hereinafter, by way of example, with reference to the following embodiment and the accompanying drawing. The only FIGURE shows schematically a piece of garment comprising dry electrodes for use with a monitoring system for monitoring physiological parameters of a recipient.

As a garment according to the invention, a sleeveless T-shirt is used, the shirt 1 fitting over the torso of a recipient (not shown). Three dry electrodes 2, 3, 4 are attached to the shirt 1. Together with a processing unit 5 the three electrodes 2, 3, 4 form an ECG monitoring system for monitoring electrical parameters of the heart. The system is used in the investigation of heart disease, especially for home use.

Each electrode 2, 3, 4 has a working surface (not shown in detail) which is to be brought into contact with the recipient's skin. The electrodes 2, 3, 4 are integrated into the garment by sewing and automatically positioned at a point of close skin contact, if the shirt is worn by the recipient.

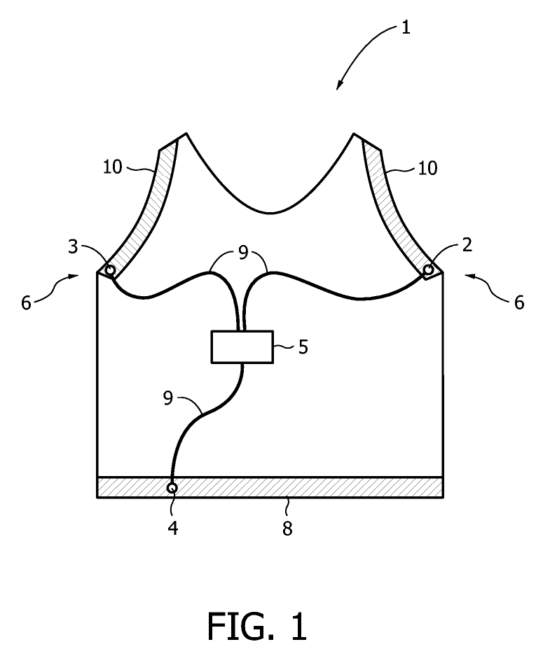

The system is adapted to capture unipolar ECG chest lead potentials by means of two ECG measuring electrodes 2, 3. Furthermore, the system requires a central reference signal, provided by a reference electrode 4. Accordingly, two dry ECG measuring electrodes 2, 3 are sewed into the textile border of the shirt 1 near the lower ends 6 of the armpit areas 10. The electrodes 2, 3 are arranged opposite each other on both sides of the chest. The electrodes 2, 3 could also be arranged at another position within the hatched areas of the shirt 1, as this area has good skin contact. However, the position at the lower ends 6 of the armpit areas 10 has been found optimal. The dry ECG reference electrode 4 is sewed into the textile border 8 at the bottom of the shirt 1, where sufficient skin contact is ensured as well. The possible electrode area for the reference electrode 4 is marked cross-hatched in FIG. 1 and comprises the complete textile border 8 at the bottom of the shirt.

The shirt 1 is normally worn and has close skin contact at the electrode positions. In the armpits of the shirt elastic or rubber springs (not shown) are used to ensure said close skin contact. The processing unit 5 and cables 9 for connecting the two measuring electrodes 2, 3 and the reference electrode 4 with the processing unit 5 are shown for illustrative purposes only. The processing unit 5 is adapted for receiving, monitoring, processing and optionally generating output messages to the recipient. The processing unit 5 is integrated into the shirt 1 as well. Alternatively, the processing unit 5 is located remotely. In the latter case, the electrodes 2, 3, 4 and the processing unit 5 comprise wireless communication means for data transmission.

It will be evident to those skilled in the art that the invention is not limited to the details of the foregoing illustrative embodiment, and that the present invention may be embodied in other specific forms without departing from the spirit or essential attributes thereof. The present embodiment is therefore to be considered in all respects as illustrative and not restrictive, the scope of the invention being indicated by the appended claims rather than by the foregoing description, and all changes which come within the meaning and range of equivalency of the claims are therefore intended to be embraced therein. It will furthermore be evident that the word “comprising” does not exclude other elements or steps, that the words “a” or “an” do not exclude a plurality, and that a single element, such as a computer system or another unit, may fulfil the functions of several means recited in the claims. Any reference signs in the claims shall not be construed as limiting the claim concerned.

### REFERENCE LIST

1 shirt
2 electrode
3 electrode
4 electrode
5 processing unit
6 lower ends
7 free
8 textile border
9 cables
10 armpit areas

1. A garment comprising at least one dry electrode for use with a non-invasive monitoring system for monitoring physiological parameters of a recipient, the electrode having a working surface which is to be brought into contact with the recipient's skin, wherein the working surface is automatically positioned at a point of close skin contact with a natural sweat layer present, if the garment worn by the recipient.

2. The garment as claimed in claim 1, wherein the at least one dry electrode is integrated into the garment.

3. The garment has claimed in claim 1, wherein the at least one dry electrode is located in the armpit area of the recipient, if the garment is worn.

4. A method of monitoring physiological parameters of a recipient by means of a non-invasive monitoring system using a garment comprising at least one dry electrode having a working surface which is to be brought into contact with the recipient's skin, wherein the working surface is automatically positioned at a point of close skin contact with a natural sweat layer present, if the garment is worn by the recipient.

5. The method as claimed in claim 4, wherein the at least one dry electrode is an ECG electrode and an ECG measurement is carried out.

6. The method as claimed in claim 4, wherein the at least one dry electrode is an impedance electrode and an impedance measurement is carried out.
